(19) United States
(12) Patent Application Publication
(10) Pub. No.: US 2012/0264205 A1
(43) Pub. Date: Oct. 18, 2012

(54) MICROBIAL DECOMPOSITION TREATMENT DEVICE AND ORGANIC SUBSTANCE TREATMENT UNIT

(76) Inventor: Masayuki Nakaya, Suita (JP)

(21) Appl. No.: 13/381,143

(22) PCT Filed: Aug. 12, 2011
(86) PCT No.: PCT/JP11/68444

§ 371 Date: Mar. 28, 2012

(30) Foreign Application Priority Data
Nov. 2, 2010 (JP) 2010-245936

(51) Int. Cl. C12M 1/06

(52) U.S. Cl. 435/290.2

(57) ABSTRACT

A organic substance treatment device is proposed which includes a cylindrical treatment tank (11) having a central raised portion on its bottom surface to create a step. An agitating vane unit (14) is provided on the raised portion. The vane unit is rotated counterclockwise if the device is located in the northern hemisphere of the earth to form a counterclockwise vortex in the liquid in the tank. By supplying air into the vortex, air is efficiently taken into the liquid. This increases efficiency with which organic substances is decomposed by aerobic microorganisms.

### TECHNICAL FIELD

This invention relates to a treatment unit for efficiently decomposing slurry containing organic substances.

### BACKGROUND ART

Typically, temporary toilets and wastewater treatment tanks that are used in places where there are no sewage system include a decomposition tank in which aerobic microorganisms are grown to decompose organic substances contained in wastewater. After decomposing organic substances, wastewater in wastewater treatment tanks may be discharged into e.g. rivers. In the case of temporary toilets, after decomposing organic substances, wastewater may be recycled. Patent document 1 discloses such a temporary toilet. In the organic substance decomposition tank of such a temporary toilet, in order to efficiently decompose organic substances in the tank with microorganisms, air is typically blown into the liquid in the tank, which contains microorganisms and organic substances, through an air blowing diffuser tube, thereby exposing microorganisms to air.

### PRIOR ART DOCUMENTS

### Patent Documents

Patent document 1: JP Patent Publication 2010-222869A

### SUMMARY OF THE INVENTION

### Object of the Invention

Since odor is produced from the tank when organic substances are decomposed, decomposition is carried out gradually with the tank hermetically sealed. But when the tank is hermetically sealed, oxygen is supplied only through the diffuser tube and from air in the tank above the liquid, which is insufficient for efficient decomposition of organic substances, and decomposition of organic substances occurs only at limited portions of the tank where oxygen is supplied. Thus, decomposition of organic substances is inefficient and time-consuming. One solution to this problem would be to use a larger decomposition tank. But such a large tank takes up a large installation space and handling is difficult too.

An object of the present invention is to improve the efficiency with which organic substances are decomposed by microorganisms, thereby reducing the installation space of the decomposition tank and thus reducing the size of the entire organic substance treatment unit.

### Means to Achieve the Object

In order to achieve this object, the present invention provides a microbial decomposition treatment device including a treatment tank for decomposing organic substances dispersed in water in the treatment tank utilizing microorganisms, wherein the treatment tank has a bottom including a central raised portion which is higher by 2 to 10 cm than a peripheral portion of the bottom of the treatment tank, and an agitating vane unit mounted on the central raised portion for forming a counterclockwise vortex of liquid in the tank if the treatment device is used in the northern hemisphere of the earth and for forming a clockwise vortex of liquid in the tank if the treatment device is used in the southern hemisphere of the earth, and wherein the treatment device further includes means for supplying air into the vortex.

Thus, instead of hermetically sealing the treatment tank as in the case of conventional aeration tanks, the tank is not sealed so that outer air containing sufficient oxygen can be supplied to the surface of the liquid in the tank. The agitating vane unit can be rotated in either direction so that a vortex in either direction can be formed depending on the location of the earth where the device is installed. A raised portion is provided at the center of the bottom of the tank to stabilize the vortex. Due to the synergistic effects of these three elements, oxygen can be distributed to the entire portion of the liquid in the treatment tank.

In the northern hemisphere of the earth, a counterclockwise vortex, as viewed from top, is formed because the Coriolis force due to rotation of the earth serves to accelerate a counterclockwise vortex in the northern hemisphere. Conversely, in the southern hemisphere, the agitating vane unit is rotated to form a clockwise vortex as viewed from top. The central raised portion, which is higher than the peripheral portion of the bottom of the tank, serves to prevent the downward flow at the center of the vortex from stopping in the area right under the agitating vane unit, and allows this downward flow to move toward the lower peripheral portion. When this flow approaches the wall surface, it now turns upward. Thus, a circulating flow of the liquid that circulates throughout the interior of the treatment tank can be easily and reliably formed.

Thus, a large vortex is formed stably. The center of such a large vortex is recessed to a large degree toward the agitating vane unit, so that air near the liquid surface can be more easily taken into the liquid. Air can thus be taken into the liquid by a larger amount than by a conventional aeration method. Particularly if a large treatment tank having a diameter exceeding one meter is used, it is possible to supply a sufficient amount of oxygen into the treatment tank, utilizing the circulation of liquid in the tank. Even if the treatment tank is small, i.e. 1 meter or less in diameter, or if the tank has to be installed at a location where its top is closed by another device, a sufficient amount of air can be introduced into the vortex by providing an air intake fan for blowing air onto the liquid surface or further installing a duct through which outer air can be supplied to the fan. Air (oxygen) taken into the liquid through the center of the vortex can be readily distributed throughout the liquid in the treatment tank by the circulating flow produced by the central raised portion (which comprises a downward flow at the center, a flow from the bottom toward the peripheral portion, an upward flow near the peripheral wall, and a flow toward the center near the liquid surface). This activates aerobic microorganisms. Even if a large-sized treatment tank is used, by blowing air into the vortex with the fan, it is possible to further accelerate decomposition.

In order to further accelerate the circulating flow, the treatment tank is preferably cylindrically shaped, or has a truncated conical shape with its bottom surface area smaller than the top surface area. In such a treatment tank, it is possible to more easily create a vortex with the agitating vane unit than in a box-shaped treatment tank. By using a truncated conical treatment tank, air can be more easily taken into the liquid because the liquid surface area is larger than the bottom surface area. This configuration is especially effective if it is difficult to form a downward flow. The larger surface area means a more stable vortex. On the other hand, a cylindrical treatment tank is more stable and can ensure a larger treating capacity.

If an air intake duct and an air intake fan are used to introduce outer air into the tank, it is preferable to further provide an exhaust duct for exhausting the same amount of air that is fed into the tank by the air intake fan, and an exhaust fan attached to the exhaust duct and operatively associated with the air intake fan for exhausting air through the exhaust duct. With this arrangement, it is possible to quickly introduce fresh air into the liquid without creating a pressure difference. If the treatment tank has to be located at such a position that its top is closed, the tank is preferably provided with an air intake fan extending diagonally from one point of the outer periphery of the tank near its top end, and an exhaust fan for expelling the same amount of air that is fed into the tank. If the air intake fan can be installed over the treatment tank, the air intake fan is preferably arranged such that its air is blown from the fan toward the center of the vortex formed by the agitating vane unit so that oxygen can be taken in most efficiently. In this case, the position where the exhaust fan is installed is not specifically limited.

The agitating vane unit, and the optional air intake fan and the exhaust fan do not have to be always operated, and may be operated intermittently. But they should be operated simultaneously. Otherwise, the efficiency with which oxygen is taken in deteriorates. By setting these members so as to be operated intermittently, it is possible to reduce power consumption, and also it is possible to reduce the quantity of solar batteries if solar batteries are used to power these members.

The treatment device according to this invention allows aerobic microorganisms to actively treat organic substances throughout the treatment tank. This makes it possible to manufacture an organic substance treatment unit in the form of a module comprising an evaporation tank for evaporating the water content from the treated water, a liquefier for collecting and liquefying the evaporated water content, and the treatment tank according to the present invention and smaller in the entire volume than conventional such treatment units. Depending on the size of the unit and the amount of air that has to be taken in, the air intake fan and the exhaust fan may be mounted in the organic substance treatment unit. The unit may further include a freshwater tank for storing liquefied water and supplying it to outside. This compact organic substance treatment unit can be easily installed in a source of wastewater containing organic substances such as a household wastewater source or an outdoor temporary toilet. If used for temporary toilets, since the treatment tank according to the present invention has a large capacity to decompose organic substances, a single such treatment unit can sufficiently treat organic substances from a plurality of temporary toilets.

Since the treatment device according to the invention can efficiently treat organic substances, large amounts of wastewater may be put into the tank at a time. Thus, the treatment device is preferably provided with a mechanism for adjusting the amount of liquid in the tank to a suitable level. For example, such a mechanism may comprise a first fluid level adjusting pipe extending upwardly from an intake port located in the treatment tank such that its highest point is located at a predetermined height of the treatment tank, and an adjusting tank for storing overflowing treated water and left at rest to allow solid contents to settle. In this case, a filter is preferably provided at the intake port of the first fluid level adjusting pipe to prevent excessive escape of microorganisms.

### Advantages of the Invention

The treatment device according to the present invention is extremely compact in size, and still can treat organic substances with high efficiency. The treatment device can thus be formed into a module for circulating water. Such a module can be easily installed in a wastewater source, and thus can be used for various wastewater sources. Since decomposition of organic substances occurs quickly throughout the entire treatment tank, odor originating from organic substances scarcely leaves the tank through the air intake or exhaust duct. Even if the top of the treatment tank is open, odor practically disappears within half an hour after organic substances have been put into the tank. This is not only because organic substances are quickly decomposed, but also because oxygen near the fluid surface tends to be taken into the liquid when a vortex forms.

The vertical circulating flow which also rotates in a vortex prevents accumulation of sludge on the bottom of the treatment tank as in the case of a conventional aeration type treatment tank. By the provision of the central raised portion, even if a solid mass of undecomposed organic substances drops onto the bottom of the tank immediately after organic substances have been put into the tank, the fluid flow toward the peripheral portion prevents such a mass of organic substances from getting tangled with the agitating vane unit, thus stopping the agitating vane unit.

Although dependent upon the size of the treatment unit, the treatment according to the present invention substantially shows a BOD load of 1000 ppm or over per liter. Compared to the BOD load of 300 ppm per liter in a conventional activated sludge method in which air is supplied by aeration, the treatment device according to the present invention has a capacity to treat organic substances that is more than three times larger than with the conventional method.

### BRIEF DESCRIPTION OF THE DRAWINGS

FIG. 1 schematically shows a treatment device according to a first embodiment of the present invention.

FIG. 2 schematically shows the treatment device of the first embodiment and peripheral devices directly connected to the treatment device.

FIG. 3 shows how wastewater is supplied into the treatment device and how the wastewater is treated in the treatment device and in the later stage.

FIG. 4 shows the entire circulation mechanism including the treatment device.

FIG. 5 schematically shows an organic substance decomposition unit embodying the present invention.

FIG. 6(a) schematically shows a treatment device according to a second embodiment of the present invention; and FIG. 6(b) is a sectional view taken along line A-A of FIG. 6(a).

### BEST MODE FOR EMBODYING THE INVENTION

Now the embodiments of this invention are described. This invention is directed to a treatment device including a microbial decomposition treatment tank (hereinafter referred to as “treatment tank 11”) for decomposing organic substances contained in an aqueous solution supplied using microbes, and a general-purpose treatment unit 5 using the treatment device. Aqueous solutions containing organic substances include household wastewater and wastewater from toilets. The organic substances contained are preferably biodegradable ones. As used herein, it is to be understood that slurry containing undissolved organic substances is one of “aqueous solutions”.

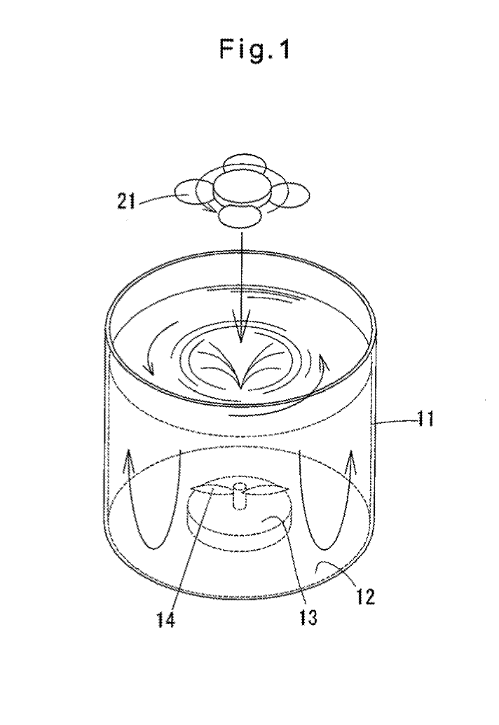

FIG. 1 schematically shows the treatment device of this embodiment. The treatment tank 11 is a cylindrical member and has a step forming a raised portion 13 at the center of the inner bottom surface thereof which is higher than the peripheral portion 12 of the inner bottom surface. An agitating vane unit 14 is supported on the raised portion 13. An air intake fan 21 is provided right over the treatment tank 11 for blowing air downwardly. Aerobic microorganisms are grown in the liquid in the treatment tank 11. They gradually decompose oxygen and organic substances that are fed into the tank.

The fan 21 and the agitating vane unit 14 are operatively coupled to each other so that they can be rotated simultaneously. When air is blown against the liquid surface by the fan 21, the agitating vane unit 14 rotates, forming a vortex in the center of the liquid, thus drawing air blown from the fan 21 into the liquid. Taking into consideration the rotational direction of the earth, the agitating vane unit 14 is rotated counterclockwise as viewed from top if used in the northern hemisphere, and clockwise if used in the southern hemisphere so as to effectively form as strong and large a whirling current as possible. The vortex causes a downward flow of the liquid at the central portion. The downward flow reaches the central raised portion 13 and moves efficiently along the step toward the peripheral portion 12. Once this flow reaches the peripheral portion 12, it now rises along the side wall. Once this upward flow reaches the liquid surface, it now flows toward the center of the vortex. The liquid thus produces a strong circulating current, which transports air drawn into the liquid, as well as oxygen separating from the air, throughout the liquid. The preferable rotational speed of the agitating vane unit 14 varies with the size of the treatment tank 11 but is determined such that the vortex has a clear eye, i.e. a hole in the liquid having a sufficient depth. Such a current allows sufficient air to be drawn into the liquid.

In order to reliably and clearly form the circulating current, the height of the step between the peripheral portion 12 and the central raised portion 13 has to be 2 cm or higher. Otherwise, it would be difficult to reliably and clearly form the circulating current. But this step has to be not more than 10 cm high. If it is higher than 10 cm, while the sufficient circulating current may form, liquid may get stuck near such a high step to an unignorable degree. The diameter of the central raised portion 13 is preferably between one-fourth and one-half, especially preferably about one-third, of the diameter of the bottom of the tank. That is, the area of the, central raised portion 13 is preferably between one-sixteenth and one-fourth, especially preferably about one-ninth, of the area of the bottom of the tank. The current induced by such a step not promotes transportation of air throughout the treatment tank 11, but also allows any mass of organic substances toward the peripheral portion, thus preventing such mass from directly hitting the agitating vane unit 14. This prevents the agitating vane unit 14 from stopping by getting clogged with a mass of organic substances.

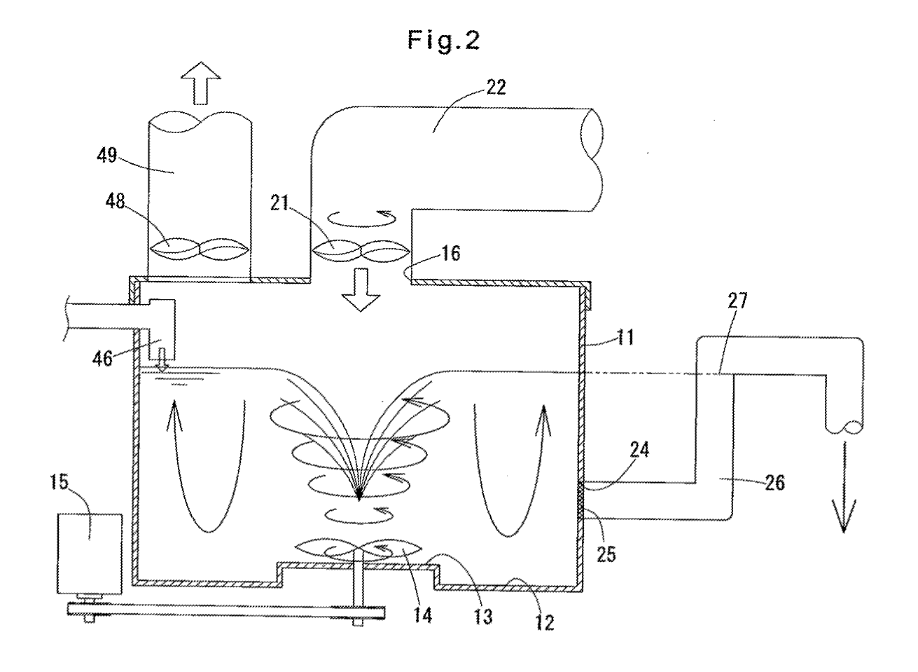

FIG. 2 schematically shows peripheral portions of the treatment tank 11. The fan 21 is connected to a duct 22 through which outer air containing sufficient amounts of oxygen can be supplied to the fan 21 when the fan 21 is on. A shutter mechanism may be provided in any portion of the duct 22 to selectively open and close the duct. But since decomposition progresses quickly in the treatment tank 11 according to the present invention, odor will barely leak even without such a shutter.

An air exhaust fan 48 is provided over the peripheral portion 12 of the treatment tank 11 which, in cooperation with the air intake fan 21, exhausts air in the treatment tank 11 into an exhaust duct 49 communicating with the outside of the tank 11.

The agitating vane unit 14 is driven by a motor 15 for the agitator, which is provided beside the treatment tank 11 in the example of FIG. 2. But in order to prevent exposure to water vapor from an evaporation tank 35, which is described later, the motor 15 is preferably provided in a sealed state over the treatment tank 11 where water vapor from the evaporation tank 35 does not reach. Preferably, the motor 15 for the agitator is also operatively coupled to the air intake fan 21 and the air exhaust fan 48. The diameter of the agitating vane unit 14 is preferably not less than one-fourth, and not more than one-half, of the inner diameter of the peripheral portion 12 of the treatment tank 11 so that a vertical vortex forms easily. Especially if the diameter of the agitating vane unit 14 is about one-third of the diameter of the peripheral portion 12, a vertical vortex forms most easily.

A wastewater input port 46 is formed in the wall of the treatment tank 11 at its position higher than the predetermined fluid level. An aqueous solution containing organic substances from an external source is fed through the port 46 and dropped into the liquid in the tank 11.

An intake port 24 is formed in the wall of the treatment tank 11 at its position lower than the predetermined fluid level through which treated liquid is discharged. A filter 25 is provided at the intake port 24 to prevent excessive leakage of undecomposed substances and microorganisms in the liquid. The intake port 24 has to be provided at a position lower than the predetermined fluid level, preferably at an intermediate position other than the upper one-third and lower one-third of the wall from the bottom of the tank to the fluid level. If the intake port 24 is located too near to the fluid level, undecomposed substances floating on the liquid are more likely to be discharged through the port 24. If the intake port 24 is located too near to the bottom of the tank, the intake port 24 could be clogged with deposits on the bottom of the tank.

A first fluid level adjusting pipe 26 is connected to the intake port 24 for adjusting the fluid level in the treatment tank 11 to a height equal to or lower than the predetermined fluid level. The first fluid level adjusting pipe 26 has its inlet connected to the intake port 24 and extends upward from the intake port 24. The highest point 27 of the lower inner periphery of the first fluid level adjusting pipe 26 is the maximum height of the predetermined fluid level in the treatment tank 11. If the fluid level exceeds the maximum height, liquid overflows the pipe 26 and drops into an adjusting tank 31 provided upstream of the first fluid level adjusting pipe 26.

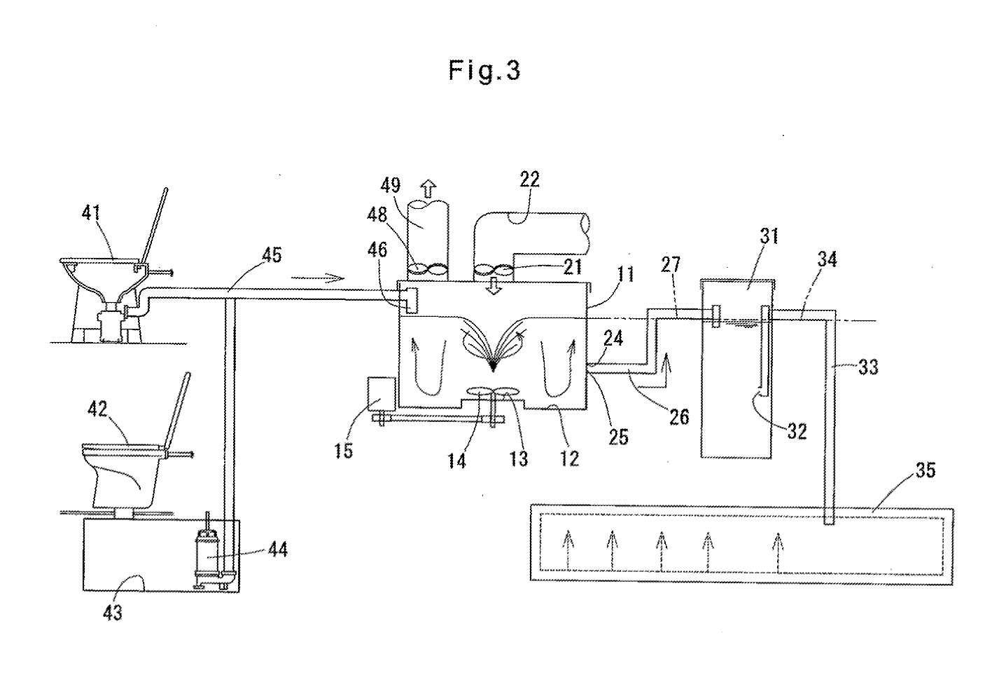

FIG. 3 schematically shows the treatment tank as well as other elements of the treatment unit according to the invention. In the adjusting tank 31, the aqueous solution containing organic substances which has been treated and supplied through the first fluid level adjusting pipe 26 (hereinafter referred to as “treated water”) is left at rest to allow solid contents to settle. A second fluid level adjusting pipe 33 is provided such that its intake port 32 is submerged in the liquid in the tank 31 so as to drop any excess liquid that exceeds the predetermined fluid level in the tank 31 into the next evaporation tank 35, thereby restricting the fluid level in the tank 31. Thus, the highest point of the lower inner periphery of the second fluid level adjusting pipe 33 is the maximum height of the predetermined fluid level in the adjusting tank 31.

The evaporation tank 35 is located below the treatment tank 11 and the adjusting tank 31, and has a large horizontal area to ensure a large surface area of the treated water that has dropped into the tank 35 through the second fluid level adjusting pipe 33. This allows evaporation of only the water content of the treated water. If this treatment unit is used in a region where the wintertime temperature drops to 20° C. or below, the evaporation tank 35 should be provided with a heater so that the heater can accelerate evaporation.

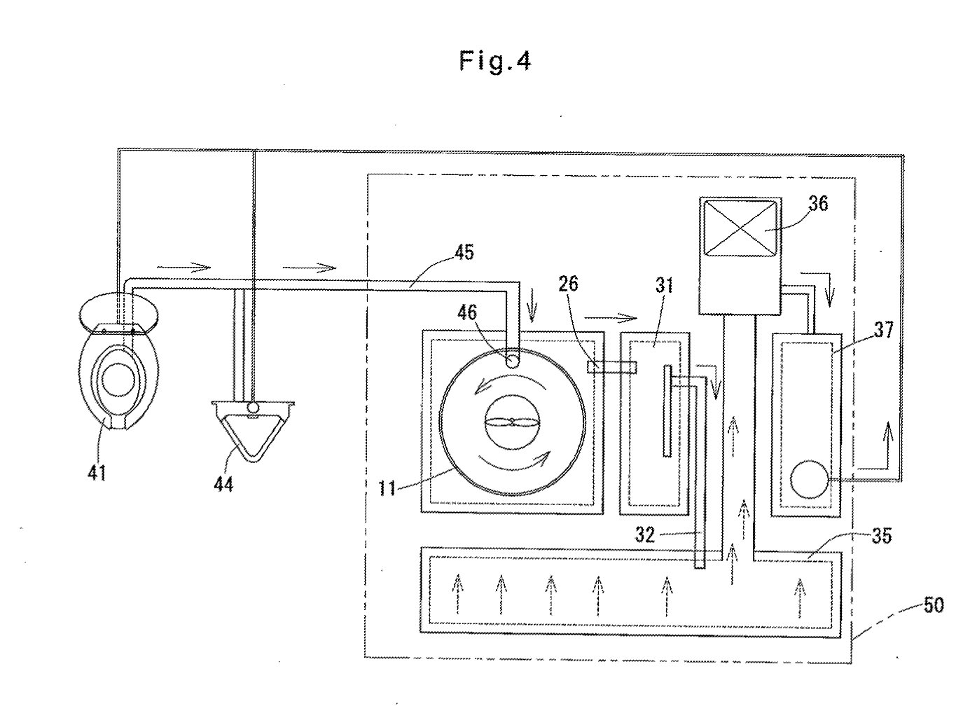

The later flows are shown in FIG. 4. The water content evaporated from the evaporation tank 35 is collected by a liquefier 36 and liquefied. The thus liquefied water, which is pure fresh water, is stored in a fresh water tank 37, and is supplied, when necessary, to external devices that need water.

By way of example, fresh water in the tank 37 is supplied to toilet facilities 41 and 42 and used as flush water and for cleaning hands. Water used in the toilet facilities is fed directly, or fed after temporarily stored in a wastewater tank 43, into the treatment tank 11 through a wastewater pipe 45 and through the wastewater input port 46 by means of a wastewater pump 44 as an aqueous solution containing organic substances. Thus, the treatment unit of the present invention constitutes a stand-alone water circulation system which can complete water recycling independently of conventional water and sewer services. For example, the treatment unit of the invention can be used as a stand-alone toilet with a flushing function.

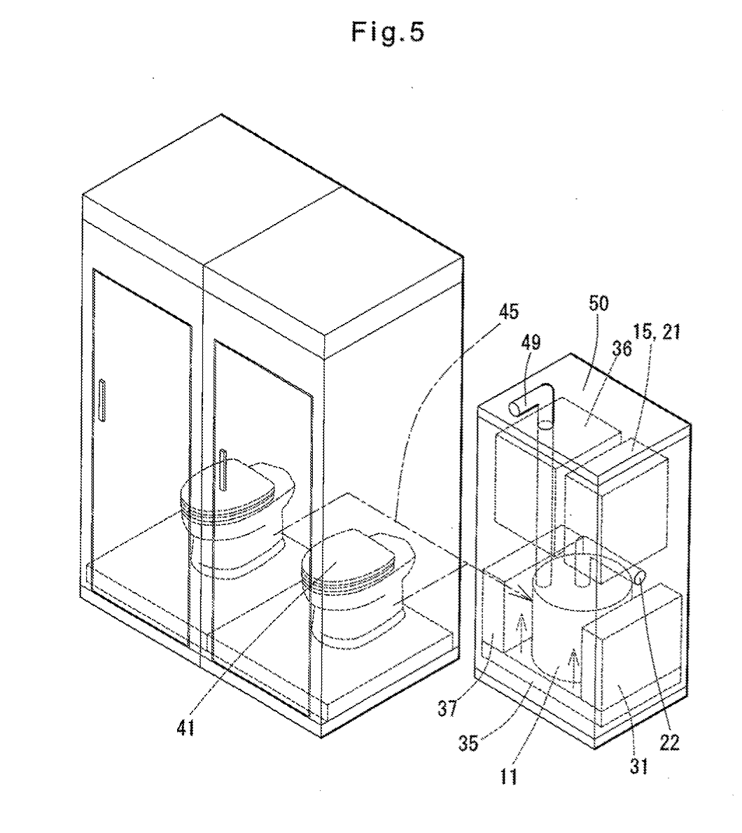

While in the example shown, the treatment unit 50 including the treatment tank according to the present invention is used as a toilet, it may be in the form of a module such as the one shown in FIG. 5, which contains the treatment tank 11, adjusting tank 31, evaporation tank 35, liquefier 36, fresh water tank 37, pipes connecting these elements together, and driving units including the motor 15 for the agitator, and the motor for the fan 21. On one side of the module, the outlet of the exhaust duct 49 opens to the outside. To assemble this module, the treatment tank 11, adjusting tank 31 and fresh water tank 37 are placed on top of the evaporation tank 35 so that water vapor evaporated from the evaporation tank 35 flows through the outside of the treatment tank 11 and is collected in the liquefier 36, which is located above. The module may further includes the heater used in a cold region to heat the evaporation tank 35, a timer mechanism for adjusting the timing of actuating the fan and the agitating vane unit, and/or a solar cell as a power source. A single such module can be used to purify wastewater from a plurality of toilets as shown in FIG. 5.

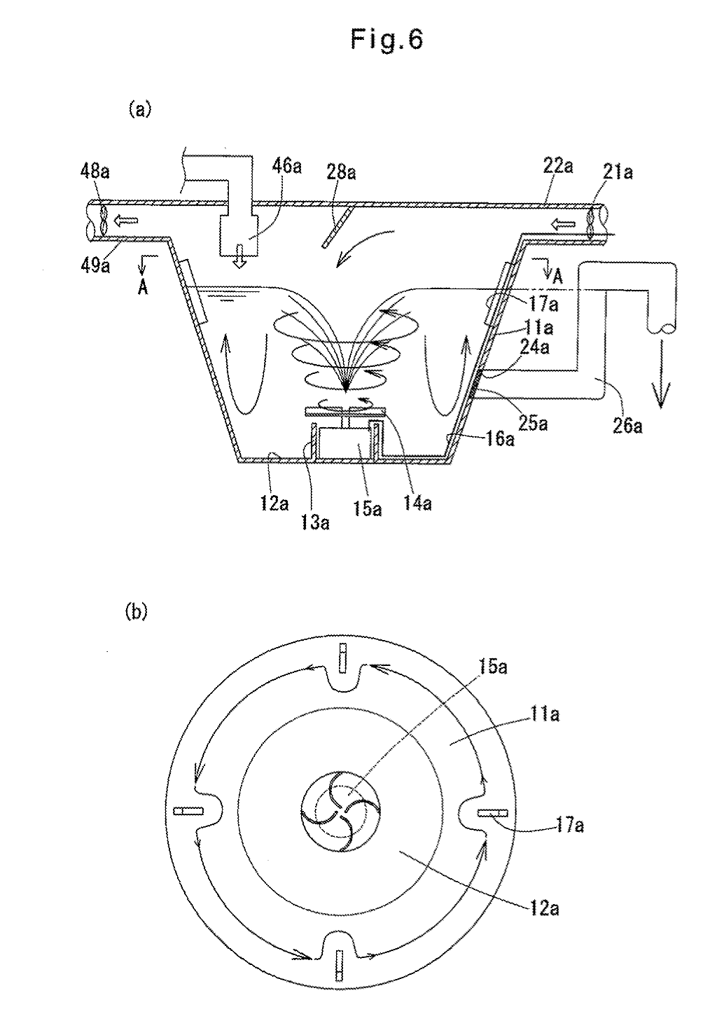

An organic substance treatment device according to another embodiment is now described with reference to FIGS. 6(a) and 6(b).

FIG. 6(a) is a sectional view of the treatment tank 11a as viewed from one side thereof. FIG. 6(b) is a sectional top plan view taken along line A-A of FIG. 6(a), which corresponds to a top plan view of the interior of the treatment tank 11a with its lid removed. The peripheral wall of the treatment tank 11a has a truncated conical shape with its top end having a larger sectional area than its bottom end.

The tank 11a has a circumferential peripheral wall 13a at the center of its inner bottom surface in which a motor 15a for an agitator, which rotates an agitating vane unit 14a, is mounted. The top edge of the peripheral wall 13a is higher by 2 to 10 cm than the peripheral portion 12a. The agitating vane unit 14a comprises a rotary disk and vanes extending vertically from the disk so as to be convex in the rotational direction. The vane unit is rotated counterclockwise as viewed from top of the treatment unit if the treatment unit is installed in the northern hemisphere of the earth, and rotated clockwise if the treatment unit is installed in the southern hemisphere. The top surface of the motor 15a for the agitator is higher by 2 to 10 cm than the bottom of the peripheral wall 13a, and thus higher than the peripheral portion 12a in the same manner as the central raised portion of the first embodiment. Preferably, the motor 15a for the agitator has a cylindrical outer shape so that the motor can be easily mounted and held in position in the peripheral wall 13a. With this arrangement, a downward vortex formed by the agitating vane unit 14a flows downward toward the peripheral portion 12a, thus forming smooth circulation of the entire liquid in the tank.

The diameter of the disk of the agitating vane unit 14a is preferably about one-fourth to one-half of the diameter of the bottom of the treatment tank 11a, and more preferably about one-third of the bottom diameter of the treatment tank for most efficient rotation of the vortex. The vanes of the agitating vane unit, which extend vertically from the disk and directly agitate the liquid, preferably extend from the central area of the disk to the area near the outer periphery of the disk. The preferable rotational speed of the agitating vane unit 14 is determined such that the vortex has a clear eye, i.e. a hole in the liquid having a sufficient depth.

The agitating vane unit 14a, which comprises the disk and the curved vanes extending vertically from the disk, is preferable to the propeller-shaped agitating vane unit 14 of the first embodiment in that the former can form a larger vortex. Since the vanes are convex in the rotational direction, hydraulic pressure on the agitating vane unit 14a decreases, which in turn reduces the load on the motor. Since the motor 15a for the agitator is mounted in the treatment tank 11a, heat generated from the motor can be used to heat the liquid in the treatment tank 11a. Thus, in a moderately cold region too, it may be possible to sufficiently activate microorganisms in the treatment tank for treatment of the liquid in the tank with the heat from the motor only, without using a separate heater.

There is no hole in the portion of the bottom of the tank surrounded by the peripheral wall 13a. Instead, power is supplied to the motor 15a for the agitator from outside through a cable 16a which extends along the peripheral wall of the treatment tank 11a to the outside of the tank 11a. With this arrangement, since it is not necessary to form a hole in the portion of the treatment tank 11a that is in contact with the liquid in the tank, the liquid in the tank can be sealed in a water-tight manner, which in turn minimizes the possibility of malfunction.

The peripheral wall of the treatment tank 11a has equidistantly spaced apart inwardly protruding protrusions 17a on its inner surface. The protrusions 17a are located at the same level as the standard fluid level. By providing the protrusions 17a near the fluid level, the vortex formed by the agitating vane unit 14a is disturbed only near the fluid level, which allows oxygen to be taken into the liquid even at portions remote from the center of the vortex. This further improves decomposing speed of the organic substances.

A duct 22a is connected to the outer periphery of the treatment tank 11a at its upper portion through which outer air is taken into the tank 11a. A fan 21a is mounted in the duct 22a for feeding air into the tank. Diametrically opposite to the duct 22a, an exhaust duct 49a is connected to the tank 11a which communicates with the outer air. An exhaust fan 48a mounted in the exhaust duct exhausts air. Since the exhaust duct is provided diametrically opposite to the duct 22a, air flows over the center of the vortex, so that air supplied through the duct 22a can be efficiently taken into the vortex under the pulling force produced by the vortex.

A guide 28a may be provided on the ceiling of the tank 11a at its portion right over the center of the vortex and on the straight line connecting the duct 22a and the exhaust duct 49a so as to direct the air flow from the duct 22a toward the below vortex, thereby efficiently supply a majority of air flow from the duct into the vortex. The guide 28a may be a simple flat plate or a plate having a surface curved along the intended curve of the air flow.

A wastewater input port 46a is provided near the peripheral wall of the treatment tank 11a at a position higher than the predetermined fluid level. An aqueous solution containing organic substances from an external source is fed through the port 46a and dropped into the liquid in the tank 11a. An intake port 24a is formed in the wall of the treatment tank 11a at its position lower than the predetermined fluid level through which treated liquid is discharged. A filter 25a is provided at the intake port 24a to prevent excessive leakage of undecomposed substances and microorganisms in the liquid. The intake port 24a has to be provided at a position lower than the predetermined fluid level, preferably at an intermediate position other than the upper one-third and lower one-third of the wall from the bottom of the tank to the fluid level. If the intake port 24 is located too near to the fluid level, undecomposed substances floating on the liquid are more likely to be discharged through the port 24a. If the intake port 24a is located too near to the bottom of the tank, the intake port 24a could be clogged with deposits on the bottom of the tank.

The wastewater input port 46a, the intake port 24a, and their internal structures may be identical or similar to those of the first embodiment. Thus, the predetermined fluid level is of the same height as the highest point 27a of a first fluid level adjusting pipe 26a connected to the intake port 24a.

The organic substance treatment device according to the second embodiment differs from the first embodiment in the arrangement and shapes of the treatment tank and the fan, but is similar to the first embodiment in its use and advantages. The unit of the second embodiment can be mounted in the module shown in FIGS. 3 to 5. For example, if there is no sufficient space above the treatment unit 11, 11a for providing the downwardly extending duct 22, it may be replaced with the combination of the duct 22a, which feed air in the lateral direction, and the guide 28a for directing the air flow downward. If the bottom area is insufficient to mount other devices, the treatment tank 11a can be used, which has a truncated conical shape, so that a large fluid surface area is ensured while minimizing the bottom surface area.

Also, only one or some of the other features of the second embodiment may be incorporated into the first embodiment. For example, the central raised portion of the first embodiment may be replaced by the center raised portion comprising the cylindrical motor 15a for the agitator and the peripheral wall 13a, it is possible to virtually prevent leakage of water. Also, protrusions, similar to the protrusions 17a may be formed on the vertically extending peripheral wall of the treatment tank 11 at its portion near the fluid level so that air can be more easily taken into the vortex.

In the above embodiments, it is necessary that water of an amount near the prescribed amount be kept in the treatment tank 11, 11a. By growing aerobic microorganisms in the water in the tank, the organic substances can be quickly and efficiently decomposed by the microorganisms using the air taken into the water. Preferably, an auxiliary agent for enzyme activity disclosed in JP Patent 3656119, which is especially suitable for use in the present invention, should be dissolved in the water in the tank by a necessary amount so as to accelerate decomposition of the organic substances by the microorganisms using the air taken into the water.

### EXAMPLES

Now the present invention is described in detail with reference to specific examples.

### <Long-Term Test>

### Example 1

Example 1 includes a cylindrical treatment tank formed of an aluminum sheet and measuring 60 cm high, and 25 cm in the bottom surface diameter (internal volume is about 120 liters), a motor, 15 cm in diameter and 5 cm high, mounted on the center of the bottom of the treatment tank, and an agitating vane unit comprising four vanes in the form of curved plates (7 cm long; each formed by diametrically cutting a pipe of 16 mm in diameter; and fixed in position so as to be angularly equidistantly spaced from each other). The agitating vane unit is rotated counterclockwise at 500 rpm (with 100 liter of water kept in the tank). The experiment was conducted indoors in Mie Prefecture, Japan. Neither a fan nor an exhaust duct was used, with the top of the tank open.

100 liter of a mixture of water and the following material was put in the treatment tank so that the mixture has a pH of 7.80, an oxidation-reduction potential (ORP) of 80 mV, and a concentration of 10000 ppm. The above material comprises mountain soil and superficial soil obtained in the Ise District, southern part of Mie Prefecture (which corresponds to the purified product obtained following the steps disclosed in JP Patent 3656119).

A test material comprising human waste (excepting paper) diluted with water was put into the above mixture every day (9.55 liters a day) from July 2010 to April 2011. On the average throughout the test period, the ORP, MLSS (Mixed Liquor Suspended Solids), pH and BOD (Biochemical Oxygen Demand) values were as follows: ORP: −50 mV; MLSS: 10000 ppm (=10 mg/liter); pH: 7.1; and BOD: 10000 ppm. From day 2, a suitable amount of water was put into the tank together with the test material so that the total amount of the liquid in the tank is 100 liters when these materials are put into the tank. Table 1 shows the mean values of MLSS, pH, ORP, water temperature, and daily reduction in the amount of liquid for each month. (At the start of the test, the MLSS value was 10000 ppm). No smell was felt from the treatment tank on any day of the test period immediately before putting a fresh supply of the test material into the tank. Also, when the interior of the treatment tank is observed immediately before putting the test material every day, no deposit of sludge was observed on the peripheral portion of the treatment tank.

TABLE 1

| | | | | | Average daily | |
|---|---|---|---|---|---|---|
| | MLSS | | ORP | Water | reduction in water | |
| | (ppm) | pH | (mV) | Temp. (° C.) | amount (liter) | Days |
| July 2010 | 10000 | 7.41 | 104 | 35.8 | 27.5 | 15 |
| August 2010 | 10000 | 7.33 | 113 | 36.2 | 27.8 | 31 |
| September 2010 | 10100 | 7.30 | 116 | 35.8 | 27.7 | 30 |
| October 2010 | 10200 | 7.24 | 121 | 35.0 | 27.6 | 31 |
| November 2010 | 10300 | 7.20 | 126 | 35.0 | 27.5 | 30 |
| December 2010 | 10400 | 7.14 | 130 | 28.8 | 27.6 | 31 |
| January 2011 | 10500 | 7.11 | 133 | 27.0 | 27.5 | 31 |
| February 2011 | 10600 | 7.09 | 136 | 27.0 | 27.5 | 28 |
| March 2011 | 10650 | 7.07 | 139 | 32.3 | 27.8 | 31 |
| April 2011 | 10800 | 7.07 | 139 | 34.3 | 28.1 | 30 |
| Total | +800 ppm | | | | 7968.8 | 288 |

During the test period of 288 days, a total of 2750.4 liters of the test material was put into the tank, and the total amount of MLSS was 27504 mg. But the final MLSS value remained +800 ppm from the starting value of 10000 ppm.

### <Short-Term Test>

### Example 2

During the above-described long-term test of Example 1, smell from the treatment tank was checked on one day of day in July 2010, after day 2 and 30 minutes after putting the test material. No smell was felt at all.

### Comparative Example 1

On the day after the day when Example 2 was conducted, when the test material was put into the tank, the agitating vane unit was temporarily rotated in the reverse direction, i.e. clockwise as seen from the top. There was an odor 30 minutes later. Then, the agitating vane unit was rotated in the original counterclockwise direction. The odor has disappeared 30 minutes later.

### <Consideration of the Central Raised Portion>

### Comparative Example 2

A treatment tank of the same size as Example 1 was prepared which has a flat bottom plate with no central raised portion, and the motor and the agitating vane unit were mounted under and on top of the bottom plate, respectively. The gap between the motor shaft and the bottom plate was water-tightly sealed. A test was conducted on this treatment tank under the same conditions as day 1 of the long-term test in Example 1. During this test, organic substances hit the agitating vane unit, thus temporarily reducing the rotating speed of the agitating vane unit. While the rotating speed of the agitating vane unit returned to normal thereafter, there was an order 30 minutes later.

### DESCRIPTION OF THE DRAWINGS

11, 11a. Treatment tank
12, 12a. Peripheral portion
13. Central raised portion
13. Peripheral wall
14, 14a. Agitating vane unit
15, 15a. Motor for an agitator
16a. Cable
17a. Protrusion
21, 21a. Fan
22, 22a. Duct
24, 24a. Intake port (connected to the first fluid level adjusting pipe)
25, 25a. Filter
26, 26a. First fluid level adjusting pipe
27, 27a. Highest point (of the first fluid level adjusting pipe)
28a. Guide
31. Adjusting tank
32. Intake port (connected to the second fluid level adjusting pipe)
33. Second fluid level adjusting pipe
34. Highest point (of the second fluid level adjusting pipe)
35. Evaporation tank
36. Liquefier
37. Fresh water tank
41, 42. Toilet
43. Wastewater tank
44. Wastewater pump
45. Wastewater pipe
46, 46a. Wastewater input port
48, 48a. Exhaust fan
49, 49a. Exhaust duct
50. Organic substance treatment unit

1. A microbial decomposition treatment device including a treatment tank for decomposing organic substances dispersed in water in the treatment tank utilizing microorganisms, wherein the treatment tank has a bottom including a central raised portion which is higher by 2 to 10 cm than a peripheral portion of the bottom of the treatment tank, and an agitating vane unit mounted on the central raised portion for forming a counterclockwise vortex of liquid in the tank if the treatment device is used in the northern hemisphere of the earth and for forming a clockwise vortex of liquid in the tank if the treatment device is used in the southern hemisphere of the earth, and wherein the treatment device further includes means for supplying air into the vortex.

2. The microbial decomposition treatment device of claim 1, wherein said means is a fan for supplying air into the treatment tank.

3. The microbial decomposition treatment device of claim 2, wherein the fan is provided over the agitating vane unit for blowing air downward.

4. The microbial decomposition treatment device of claim 1, wherein the treatment tank has a cylindrical outer shape, or a truncated conical outer shape with its bottom end surface having a smaller area than its top end surface.

5. The microbial decomposition treatment device of claim 1, wherein the fan is connected to a duct through which air can be taken into the treatment tank from outside.

6. The microbial decomposition treatment device of claim 1, wherein the inwardly protruding protrusions are formed on the inner periphery of the treatment tank for disturbing the flow of the vortex formed by the agitating vane unit near the surface of the liquid.

7. An organic substance treatment unit, comprising:
the microbial decomposition treatment device of claim 1;
an evaporation tank for evaporating the water content from the treated water which has been decomposed by microorganisms; and
a liquefier for collecting and liquefying the evaporated water content.

8. The organic substance treatment unit of claim 7, further comprising an adjusting tank for storing the treated water which has been decomposed by microorganisms; and
a first fluid level adjusting pipe having an intake port located at a lower level than a predetermined fluid level of the liquid in the treatment tank, and extending upwardly from the intake port such that the highest point thereof is located at the same level as the predetermined fluid level, whereby the fluid level of the liquid in the treatment tank is kept at a level equal to or lower than the predetermined fluid level.

9. The organic substance treatment unit of claim 8, further comprising a filter provided at the intake port of the first fluid level adjusting pipe.

10. The microbial decomposition treatment device of claim 2, wherein the treatment tank has a cylindrical outer shape, or a truncated conical outer shape with its bottom end surface having a smaller area than its top end surface.

11. The microbial decomposition treatment device of claim 3, wherein the treatment tank has a cylindrical outer shape, or a truncated conical outer shape with its bottom end surface having a smaller area than its top end surface.

12. The microbial decomposition treatment device of claim 2, wherein the fan is connected to a duct through which air can be taken into the treatment tank from outside.

13. The microbial decomposition treatment device of claim 3, wherein the fan is connected to a duct through which air can be taken into the treatment tank from outside.

14. The microbial decomposition treatment device of claim 4, wherein the fan is connected to a duct through which air can be taken into the treatment tank from outside.

15. The microbial decomposition treatment device of claim 2, wherein the inwardly protruding protrusions are formed on the inner periphery of the treatment tank for disturbing the flow of the vortex formed by the agitating vane unit near the surface of the liquid.

16. The microbial decomposition treatment device of claim 3, wherein the inwardly protruding protrusions are formed on the inner periphery of the treatment tank for disturbing the flow of the vortex formed by the agitating vane unit near the surface of the liquid.

17. The microbial decomposition treatment device of claim 4, wherein the inwardly protruding protrusions are formed on the inner periphery of the treatment tank for disturbing the flow of the vortex formed by the agitating vane unit near the surface of the liquid.

18. The microbial decomposition treatment device of claim 5, wherein the inwardly protruding protrusions are formed on the inner periphery of the treatment tank for disturbing the flow of the vortex formed by the agitating vane unit near the surface of the liquid.

19. An organic substance treatment unit, comprising:
the microbial decomposition treatment device of claim 2;
an evaporation tank for evaporating the water content from the treated water which has been decomposed by microorganisms; and
a liquefier for collecting and liquefying the evaporated water content.

20. An organic substance treatment unit, comprising:
the microbial decomposition treatment device of claim 3;
an evaporation tank for evaporating the water content from the treated water which has been decomposed by microorganisms; and
a liquefier for collecting and liquefying the evaporated water content.

21. An organic substance treatment unit, comprising:
the microbial decomposition treatment device of claim 4;
an evaporation tank for evaporating the water content from the treated water which has been decomposed by microorganisms; and
a liquefier for collecting and liquefying the evaporated water content.

22. An organic substance treatment unit, comprising:
the microbial decomposition treatment device of claim 5;
an evaporation tank for evaporating the water content from the treated water which has been decomposed by microorganisms; and
a liquefier for collecting and liquefying the evaporated water content.

23. An organic substance treatment unit, comprising:
the microbial decomposition treatment device of claim 6;
an evaporation tank for evaporating the water content from the treated water which has been decomposed by microorganisms; and
a liquefier for collecting and liquefying the evaporated water content.
